(19) United States
(12) Patent Application Publication
(10) Pub. No.: US 2018/0360463 A1
(43) Pub. Date: Dec. 20, 2018

(54) REMOTE ISCHEMIC PRECONDITIONING APPLICATION DEVICE AND METHOD

(71) Applicant: Jack Rubinstein, Akron, OH (US)

(72) Inventor: Jack Rubinstein, Akron, OH (US)

(21) Appl. No.: 16/010,162

(22) Filed: Jun. 15, 2018

Related U.S. Application Data
(60) Provisional application No. 62/520,263, filed on Jun. 15, 2017.

(51) Int. Cl. A61B 17/135; A61B 17/132

(52) U.S. Cl. CPC A61B 17/135; A61B 17/1322

(57) ABSTRACT

A remote ischemic preconditioning application device may include a first compression portion that is operable to apply a coil shaped pressure pattern around at least 360 degrees of an upper arm portion and a second compression portion that is selectively operable to apply a coil shaped pressure pattern around at least 360 degrees of a lower arm portion. The device may use remote ischemic preconditioning to: reduce blood flow in the upper and lower arm portions for at least 30 continuous minutes; and 2) increase blood flow in the patient as a result of remote ischemic preconditioning.

This application claims priority to U.S. Provisional Patent Application No. 62/520,263 entitled “USE OF RECURRENT FULL LIMB PREEMPTIVE CONDITIONING DEVICE TO REDUCE THE INCIDENCE AND SEVERITY OF ISCHEMIC HEART DISEASE” filed on Jun. 15, 2017, the contents of which are incorporated herein.

### I. BACKGROUND OF THE INVENTION

### A. Field of Invention

This invention pertains to the art of methods and apparatuses regarding Remote Ischemic Preconditioning (RIPC).

### B. Description of the Related Art

Remote ischemic preconditioning (RIPC) is a process via which short periods of sub-lethal ischemia administered to a distant organ or extremity prior to an ischemic cardiac insult results in decreased injury. The concept of RIPC has been well established in the scientific literature for over a quarter of century, though the translation of this finding to the bedside has largely been unsuccessful as the majority of trials have involved short bursts of sub-lethal ischemia via single pressure point compression in patients already suffering from ischemic heart disease (IHD).

### II. SUMMARY OF THE INVENTION

According to some embodiments of this invention, a remote ischemic preconditioning application device may be used with an associated patient having an arm with an upper arm portion and a lower arm portion. The device may comprise: a first compression portion that is selectively operable to apply a coil shaped pressure pattern around at least 360 degrees of the upper arm portion; a second compression portion that is selectively operable to apply a coil shaped pressure pattern around at least 360 degrees of the lower arm portion; and a controller that operates the first and second compression portions using remote ischemic preconditioning to: 1) reduce blood flow in the upper and lower arm portions for at least 30 continuous minutes; and 2) increase blood flow in the patient as a result of remote ischemic preconditioning.

According to some embodiments of this invention, the remote ischemic preconditioning application device may include a first compression portion that is selectively operable to apply the coil shaped pressure pattern as a generally helically shaped pressure pattern around the upper arm portion for at least 360 degrees; and a second compression portion that is selectively operable to apply the coil shaped pressure pattern as a generally helically shaped pressure pattern around the lower arm portion for at least 1440 degrees.

According to some embodiments of this invention, the remote ischemic preconditioning application device may include a first compression portion that is selectively inflatable to apply the coil shaped pressure pattern around the upper arm portion; and a second compression portion that is selectively inflatable to apply the coil shaped pressure pattern around the lower arm portion.

According to some embodiments of this invention, the remote ischemic preconditioning application device may include a first compression portion that is selectively operable to apply the coil shaped pressure pattern around the upper arm portion using only sub-occlusive pressure; and a second compression portion that is selectively operable to apply the coil shaped pressure pattern around the lower arm portion using only sub-occlusive pressure.

According to some embodiments of this invention, the remote ischemic preconditioning application device may include a first compression portion as a bicep cuff; and a controller that is operable using the bicep cuff and a pressure sensor to determine systolic and diastolic blood pressure of the associated patient's arm.

According to some embodiments of this invention, the remote ischemic preconditioning application device may include a controller that operates the first and second compression portions using remote ischemic preconditioning to reduce blood flow in the upper and lower arm portions simultaneously.

According to some embodiments of this invention, the remote ischemic preconditioning application device may include a controller that operates the first and second compression portions using remote ischemic preconditioning to reduce blood flow in the upper and lower arm portions sequentially.

According to some embodiments of this invention, a method may be used in applying remote ischemic preconditioning to an associated patient having an arm with an upper arm portion and a lower arm portion. The method may comprise the steps of: A) providing a remote ischemic preconditioning application device comprising: 1) a first compression portion that is selectively operable to apply a coil shaped pressure pattern around at least 360 degrees of the upper arm portion; 2) a second compression portion that is selectively operable to apply a coil shaped pressure pattern around at least 360 degrees of the lower arm portion; and 3) a controller that operates the first and second compression portions; B) providing the remote ischemic preconditioning application device to be operable to perform the steps of: 1) reducing blood flow in the upper and lower arm portions for at least 30 continuous minutes; and 2) increasing blood flow in the patient as a result of remote ischemic preconditioning.

According to some embodiments of this invention, the method may include the step of: reducing blood flow in the upper and lower arm portions simultaneously.

According to some embodiments of this invention, the method may include the step of: reducing blood flow in the upper and lower arm portions sequentially.

According to some embodiments of this invention, the method may include the step of: reducing blood flow in the upper and lower arm portions using only sub-occlusive pressure.

According to some embodiments of this invention, the method may include the steps of: providing the first compression portion to be selectively inflatable to apply the coil shaped pressure pattern as a generally helically shaped pressure pattern around the upper arm portion; and providing the second compression portion to be selectively inflatable to apply the coil shaped pressure pattern as a generally helically shaped pressure pattern around the lower arm portion.

According to some embodiments of this invention, the method may include the steps of: providing the first compression portion to be a bicep cuff; providing a pressure sensor; and providing the controller to be operable using the bicep cuff and the pressure sensor to determine systolic and diastolic blood pressure of the associated patient's arm.

According to some embodiments of this invention, the method may include a controller that is operable to: calculate the mean arterial pressure from the blood pressure; re-inflate the bicep cuff to achieve and maintain the mean arterial pressure; and deflate the bicep cuff after 30 continuous minutes.

According to some embodiments of this invention, a remote ischemic preconditioning application device may be used with an associated patient having an arm with an upper arm portion and a lower arm portion. The device may comprise: a bicep cuff that is selectively operable to apply a coil shaped pressure pattern around at least 360 degrees of the upper arm portion; a forearm strap that extends from the bicep cuff and that is selectively operable to apply a coil shaped pressure pattern around at least 360 degrees of the lower arm portion; and a controller that operates the bicep cuff and the forearm strap using remote ischemic preconditioning to: 1) reduce blood flow in the upper and lower arm portions using only sub-occlusive pressure; and 2) increase blood flow in the patient as a result of remote ischemic preconditioning.

According to some embodiments of this invention, a remote ischemic preconditioning application device may include a bicep cuff that is selectively operable to apply the coil shaped pressure pattern as a generally helically shaped pressure pattern around at least 720 degrees of the upper arm portion; and a forearm strap that is selectively operable to apply the coil shaped pressure pattern as a generally helically shaped pressure pattern around at least 1440 degrees of the lower arm portion.

According to some embodiments of this invention, a remote ischemic preconditioning application device may include a bicep cuff that is selectively inflatable to apply the generally helically shaped pressure pattern around the upper arm portion; and a forearm strap that is selectively inflatable to apply the generally helically shaped pressure pattern around the lower arm portion.

According to some embodiments of this invention, a remote ischemic preconditioning application device may include a bicep cuff that comprises an arm contact surface that is selectively operable to apply the generally helically shaped pressure pattern around the upper arm portion; and a forearm strap that comprises an arm contact surface that is selectively operable to apply the generally helically shaped pressure pattern around the lower arm portion. The arm contact surface of the bicep cuff may have a width of 1.0 inches or less; and the arm contact surface of the forearm strap may have a width of 1.0 inches or less.

According to some embodiments of this invention, a remote ischemic preconditioning application device may include a controller that is operable using the bicep cuff and a pressure sensor to determine systolic and diastolic blood pressure of the associated patient's arm.

According to some embodiments of this invention, a remote ischemic preconditioning application device may include a controller that operates the bicep cuff and the forearm strap using remote ischemic preconditioning to reduce blood flow in the upper and lower arm portions for at least 30 continuous minutes.

Benefits and advantages of the invention will become apparent to those skilled in the art to which it pertains upon a reading and understanding of the following detailed specification.

### III. BRIEF DESCRIPTION OF THE DRAWINGS

The invention may take physical form in certain parts and arrangement of parts, embodiments of which will be described in detail in this specification and illustrated in the accompanying drawings which form a part hereof and wherein:

FIG. 1 illustrates a human body.

FIG. 2 shows peak systolic velocity in the brachial artery of a contralateral arm in patients who used an RIPC device according to embodiments of this invention in comparison to control subjects (n=9 and 11; respectively).

FIG. 3 shows the percent change in brachial artery diameter before and after a patient's use of an RIPC device according to embodiments of this invention.

FIG. 4 shows monocyte adhesion to endothelial surface in patients who used an RIPC device according to embodiments of this invention.

FIG. 5 shows a RIPCD device according to some embodiments of this invention.

FIGS. 6A and 6B show a RIPCD device according to some embodiments of this invention.

FIG. 7 shows a RIPCD device according to some embodiments of this invention.

FIG. 8 shows a RIPCD device according to some embodiments of this invention.

FIG. 9 shows a RIPCD device according to some embodiments of this invention.

FIG. 10 shows a RIPCD device according to some embodiments of this invention.

FIG. 11 shows a RIPCD device according to some embodiments of this invention.

FIG. 12 shows a RIPCD device according to some embodiments of this invention.

FIG. 13 shows a RIPCD device according to some embodiments of this invention.

FIGS. 14A and 14B show a RIPCD device according to some embodiments of this invention.

FIG. 15 shows a RIPCD device according to some embodiments of this invention.

FIG. 16 shows a RIPCD device according to some embodiments of this invention.

FIG. 17 shows a RIPCD device according to some embodiments of this invention.

FIG. 18 shows a RIPCD device according to some embodiments of this invention.

### IV. DETAILED DESCRIPTION

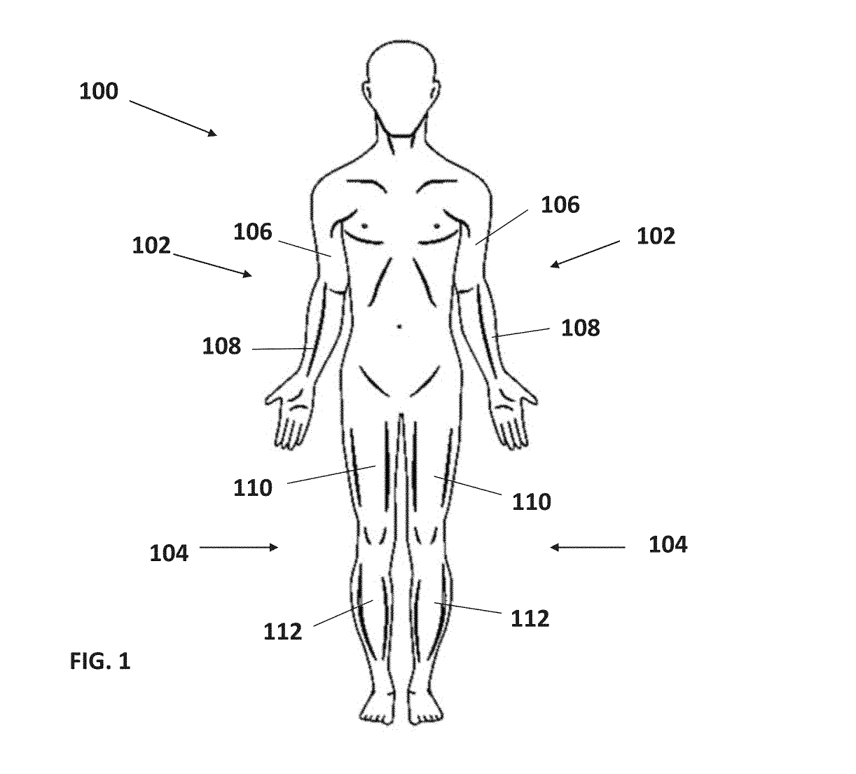

Referring now to the drawings wherein the showings are for purposes of illustrating embodiments of the invention only and not for purposes of limiting the same, FIG. 1 illustrates a patient 100, including limbs in the form of arms 102, 102, and legs 104, 104, each having upper and lower portions 106, 108, and 110, 112, respectively. While a later to be described remote ischemic preconditioning application device will be described as being applied to one of the patient's arms 102, it should be understood that in other embodiments it may be applied to one of the patient's legs 104. It should also be understood that while the remote ischemic preconditioning application device will be described as being applied to a single limb, in other embodiments in may be applied to two or more limbs in any combination chosen with the sound judgment of a person of skill in the art. Because both the upper and lower portions of the limb are used in the embodiments described, the remote ischemic preconditioning application device may be considered a “full limb” device. This does not mean that the device must contact every part of a patient's limb, it means that the device contacts and applies a compression force to at least part of the upper limb portion and part of the lower limb portion.

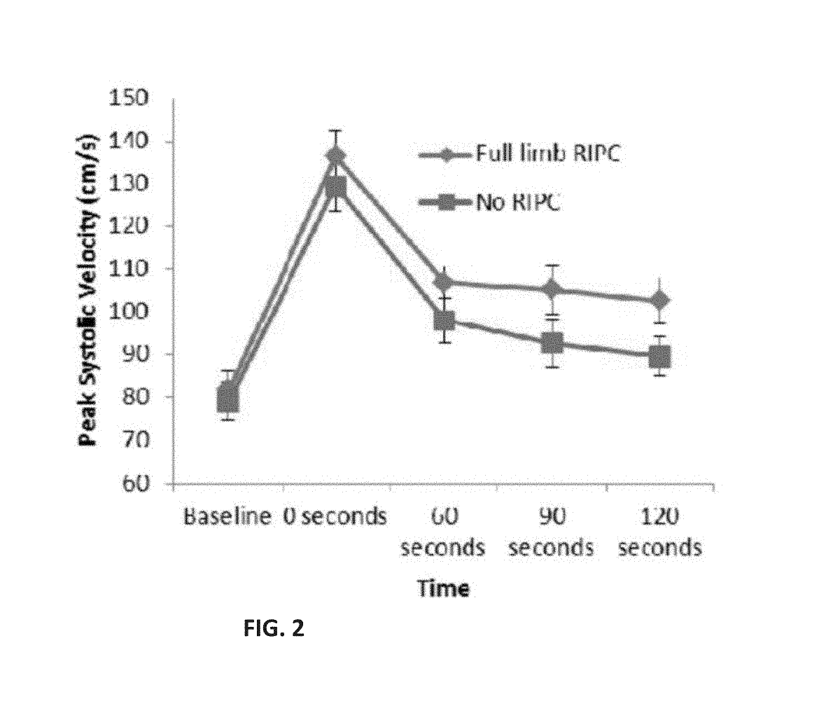

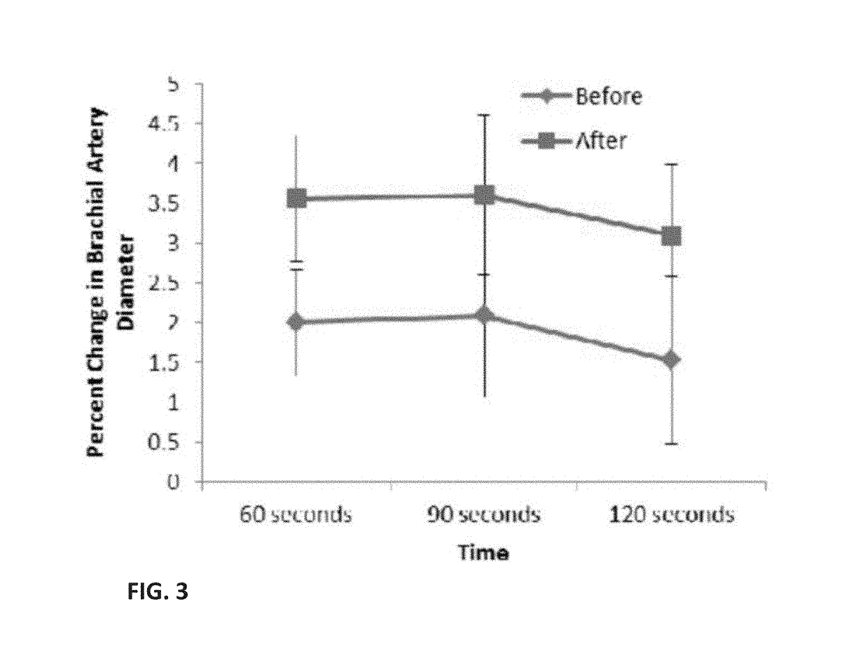

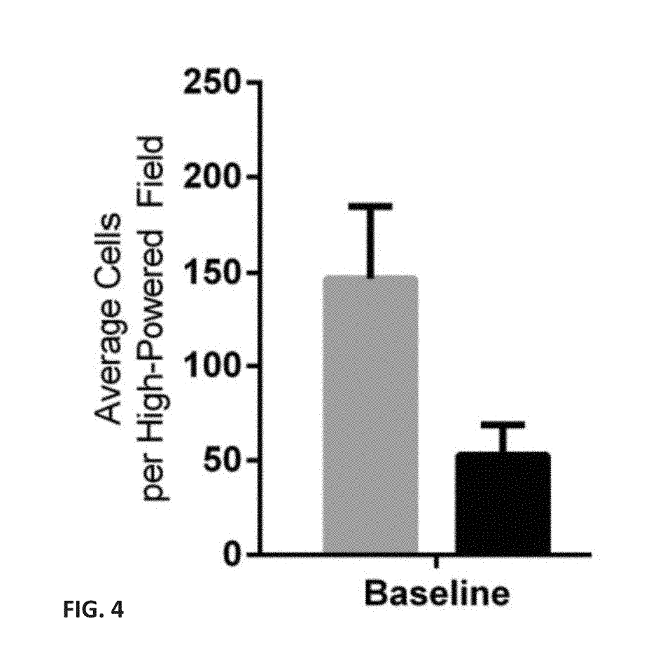

The present inventor was surprised to discover, but then established, that relatively longer, consistent, and full limb compression for at least 30 minutes in patients at risk, but not suffering from ischemic heart disease (IHD), results in beneficial remote ischemic preconditioning (RIPC) stimulus. Specifically, subjects with this type of RIPC stimulus experienced increased blood flow in the contralateral arm as measured via peak systolic velocity via ultrasound as shown in FIG. 2 as well as decreased monocyte adhesion in vitro as shown in FIG. 3. An almost two fold increase in change in brachial artery diameter in the contralateral arm acutely after a single use was detected as shown in FIG. 4.

The present inventor also discovered that numerous embodiments of remote ischemic preconditioning application devices (RIPCDs) can be used to achieve the desired beneficial RIPC results. In each case, the RIPCD includes a first compression portion that is selectively operable to apply a coil shaped pressure pattern around at least 360 degrees of the upper limb portion and a second compression portion that is selectively operable to apply a coil shaped pressure pattern around at least 360 degrees of the lower limb portion. By “coil shaped” it meant any looping pattern generally in the shape of a coil. In some non-limiting embodiments, the coil shaped pressure pattern is helical in shape.

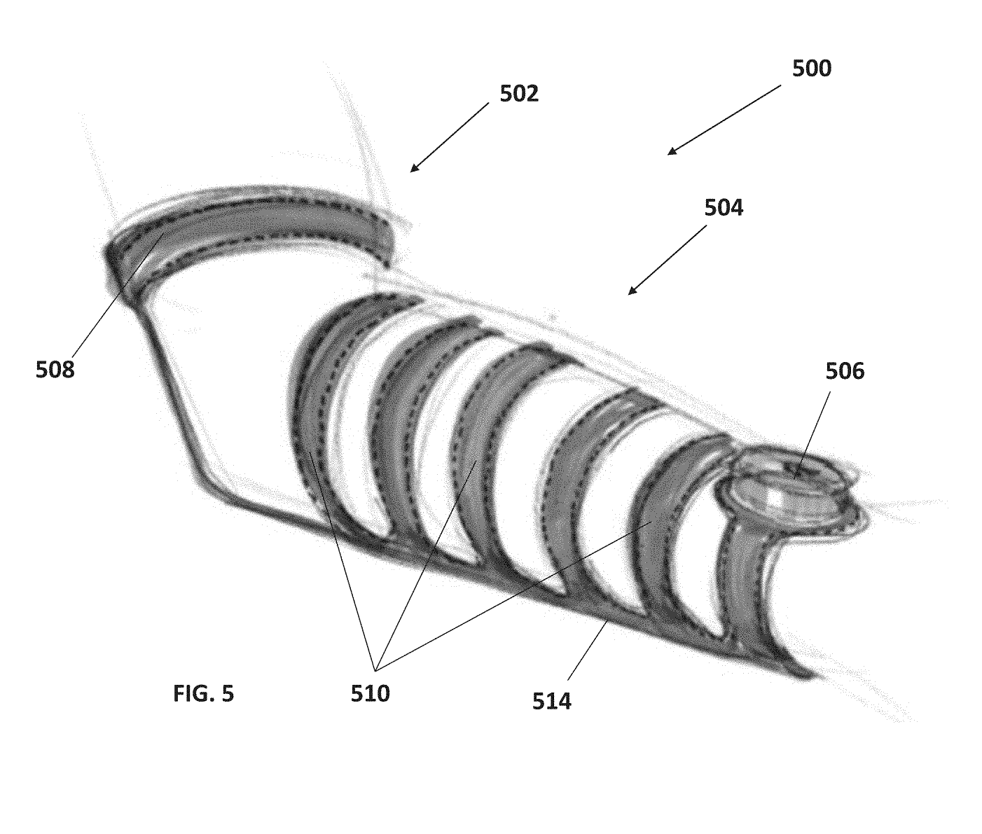

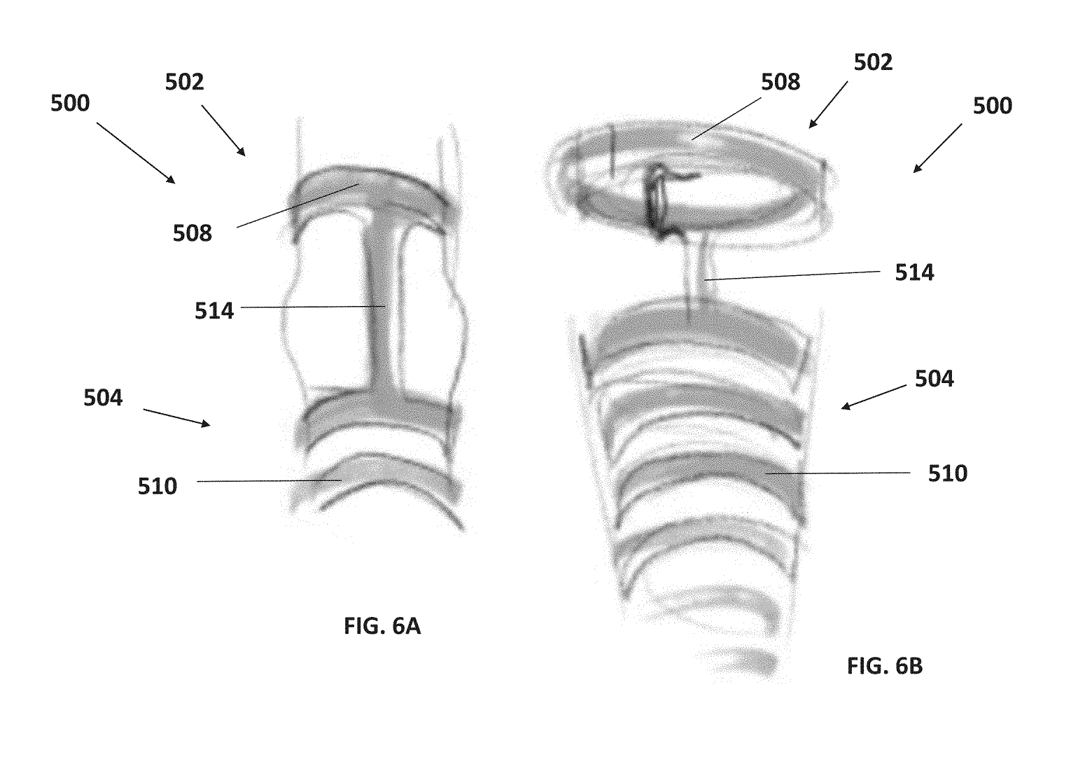

FIGS. 5, 6A and 6B show one embodiment RIPCD 500 having a first compression portion 502 that is selectively operable to apply a coil shaped pressure pattern around at least 360 degrees of the upper arm portion (the bicep portion) and a second compression portion 504 that is selectively operable to apply a coil shaped pressure pattern around at least 360 degrees of the lower arm portion (the forearm portion), as shown. For the embodiment shown, the first and second compression portions 502, 504 are each formed of straps 508, 510, respectively, that wrap around the patient's arm. The RIPCD 500 may, in some embodiments, also include a webbing portion 514 that interconnects the straps 506, 508. Such interconnection makes the RIPCD 500 a unitary device, easy to store and apply by an operator. In a more specific embodiment, the second compression portion 504 may be selectively operable to apply a coil shaped pressure pattern around at least 1440 degrees of the lower arm portion (at least four full loops). In yet another embodiment, shown, the second compression portion 504 is selectively operable to apply a coil shaped pressure pattern around at least 2160 degrees of the lower arm portion (at least six full loops). In yet another embodiment, one or both of the compression portions 502, 504 may be generally helically shaped.

With continuing reference to FIGS. 5, 6A and 6B, the RIPCD may also have a controller 506 that operates the compression portions 502, 504 using remote ischemic preconditioning in order to: 1) reduce the blood flow in the upper and lower arm portions for at least 30 continuous minutes; and 2) increase blood flow in the patient as a result of remote ischemic preconditioning. The controller 506 may be of any type chosen with the sound judgment of a person of skill in the art and may include and/or work with one or more pressure sensors positioned to determine the required pressure(s). In some embodiments a pressure sensor measures the patient's blood pressure (such as systolic and diastolic) in the patient's arm. In other embodiments, a pressure sensor measures inflation pressure created by the RIPCD. The controller 506 may be used to increase the tightness of the compression portions 502, 504 on the patient's arm. In one embodiment, the controller 506 includes a microprocessor that is programmed to perform the algorithms necessary to achieve the pressures in the limb described.

In some embodiments, the controller 506 may be used to apply the pressure in one or both of the compression portions 502, 504 using only sub-occlusive pressure. In some embodiments, the controller 508 may be used to reduce blood flow in the upper and lower arm portions 502, 504 simultaneously. In another embodiment, the controller 508 may be used to reduce blood flow in the upper and lower arm portions 502, 504 sequentially. The controller 508 may, for example, reduce blood from in the upper arm portion 502 before (or after) it reduces blood from in the lower arm portion 504. In some embodiments where inflation pressure is used, the controller is operable to: calculate the mean arterial pressure from the blood pressure; re-inflate the bicep cuff to achieve and maintain the mean arterial pressure; and deflate the bicep cuff after 30 continuous minutes

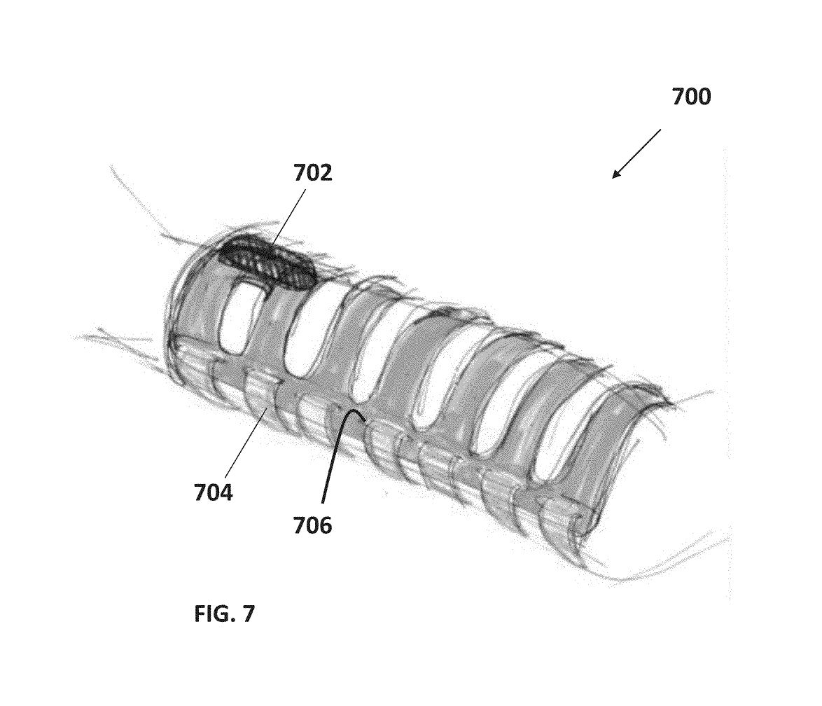

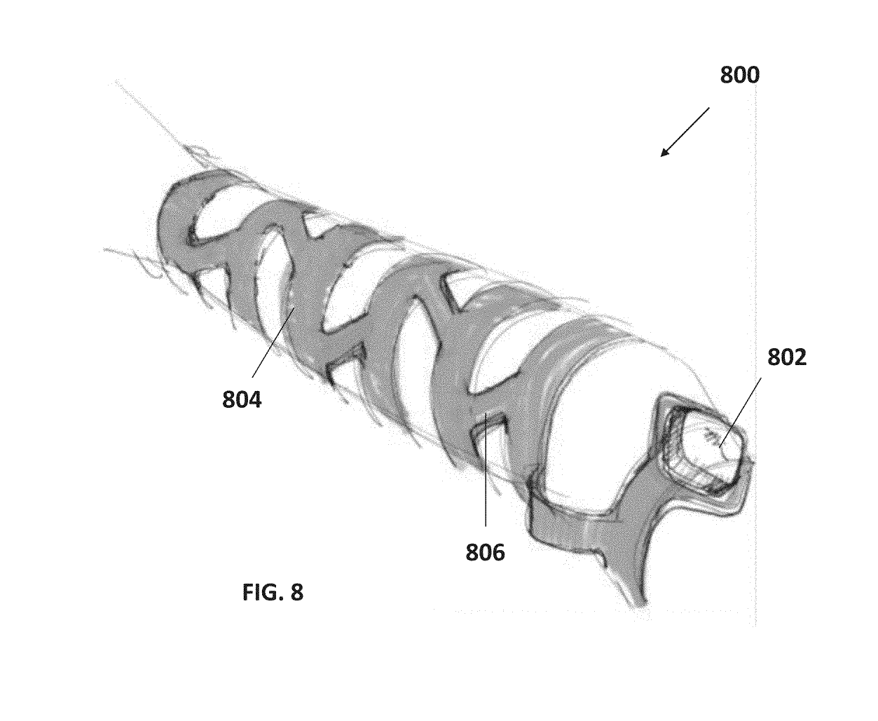

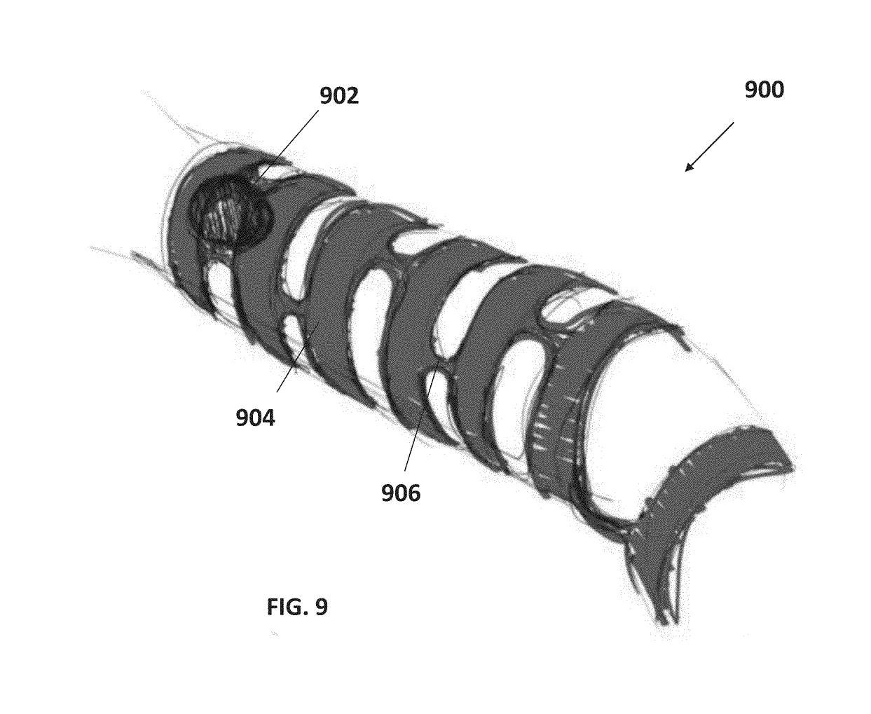

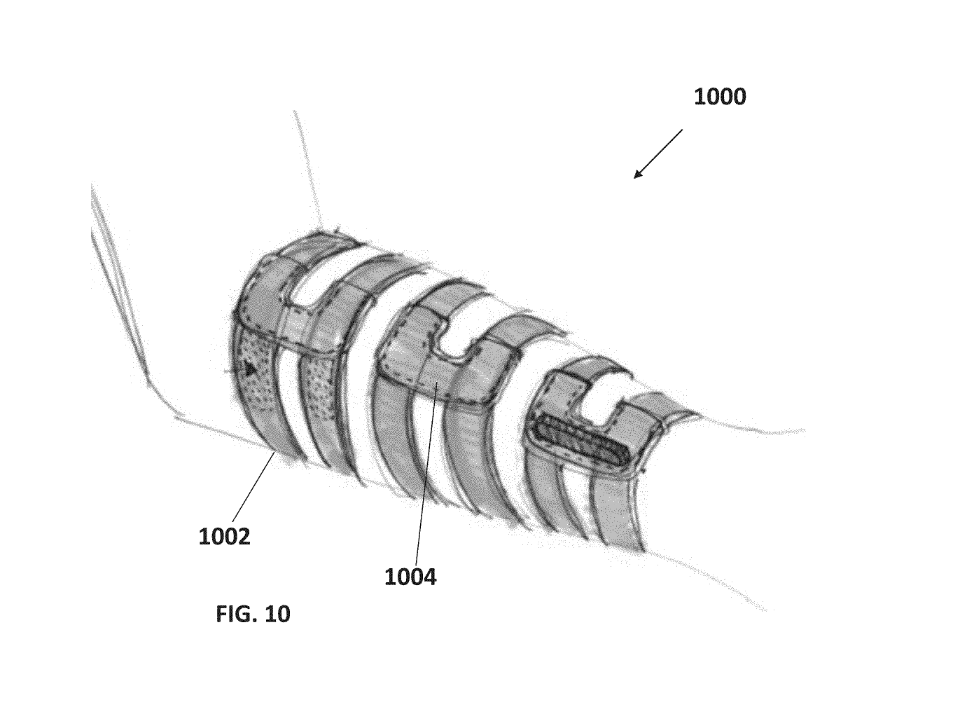

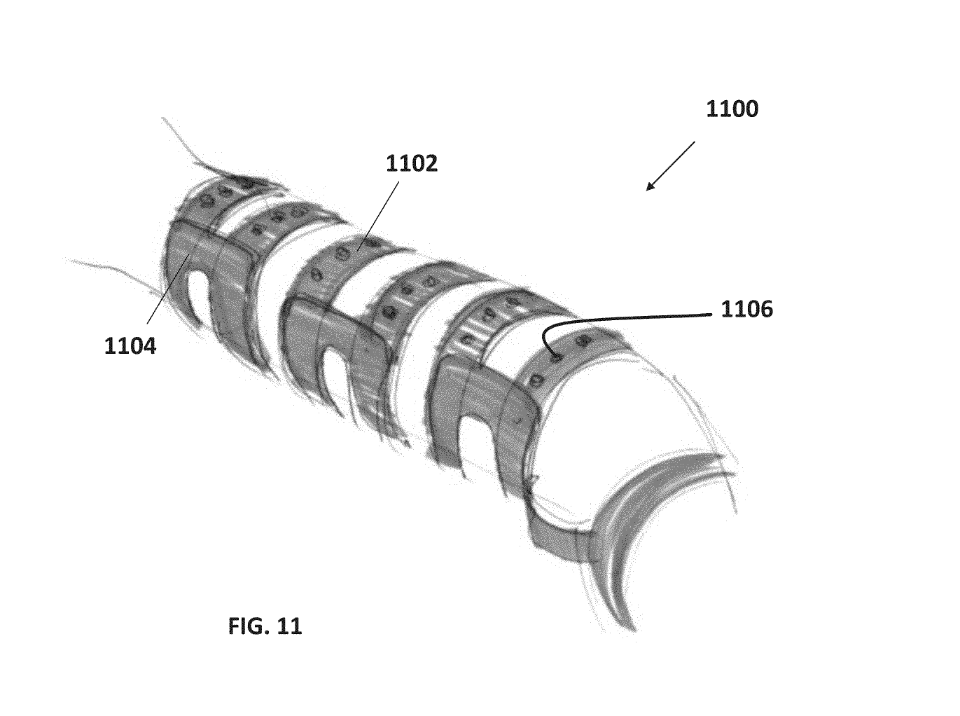

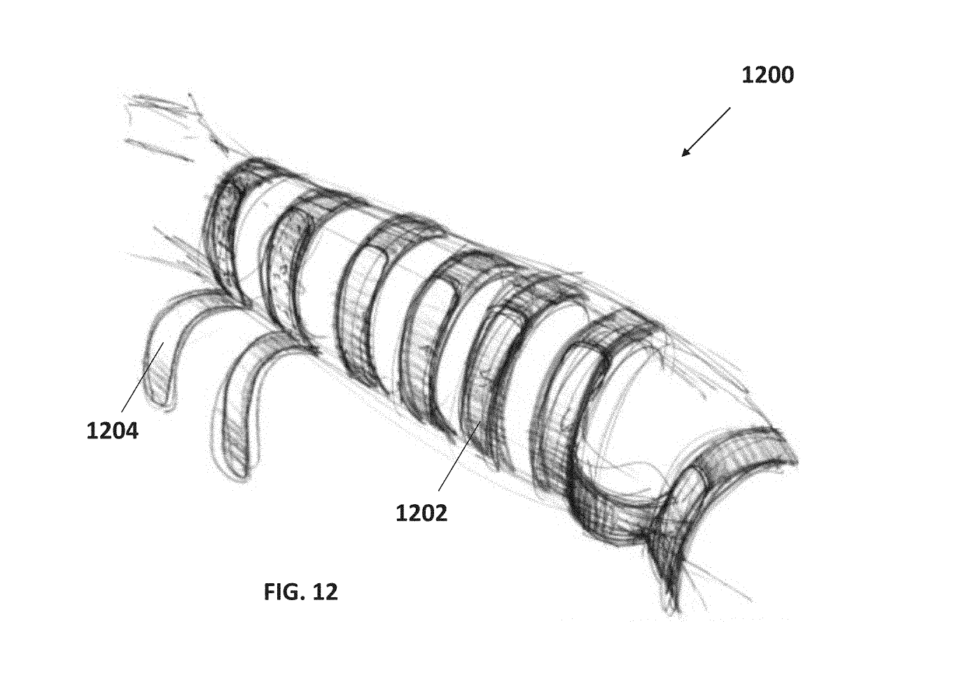

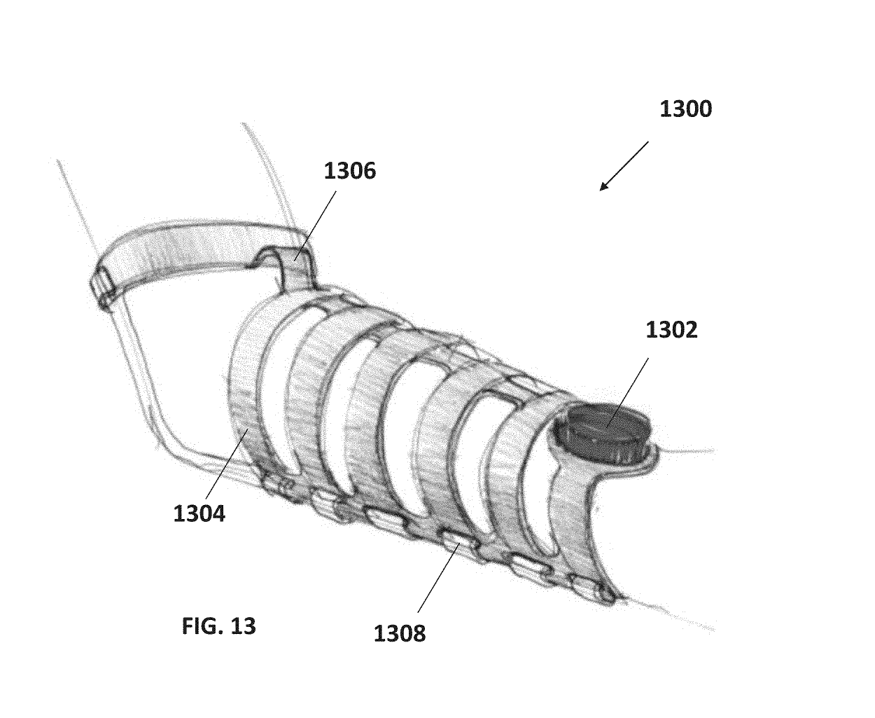

FIGS. 7-13, illustrate RIPCDs 600, 700, 800, 900, 1000, 1100, 1200 and 1300 that operate similarly to RIPCD 500 in that they include a first compression portion that is selectively operable to apply a coil shaped pressure pattern around at least 360 degrees of the upper arm portion (the bicep portion), a second compression portion that is selectively operable to apply a coil shaped pressure pattern around at least 360 degrees of the lower arm portion (the forearm portion) and a controller that operates the compression portions using remote ischemic preconditioning in order to: 1) reduce the blood flow in the upper and lower arm portions for at least 30 continuous minutes; and 2) increase blood flow in the patient as a result of remote ischemic preconditioning. RIPCD 700, see FIG. 7, includes controller 702 and multiple straps 704 that can be manually inserted through corresponding openings 706, as shown, to initially attached the RIPCD 700 to the patient's arm. RIPCDs 800 and 900, see FIGS. 8-9, each include a controller 802, 902 and multiple straps 804, 904 that are interconnected with webbing portions 806, 906 respectively, but the specific pattern varies, as shown. RIPCDs 1000 and 1100, see FIGS. 10-11, include straps 1002, 1102 that can be held together with fasteners 1004, 1104. The fasteners 1004 may be hook and loop fasteners that selectively attach to the straps 1002. Fasteners 1104 may use tabs received in holes 1106 formed in the straps 1102. RIPCD 1200, see FIG. 12, includes multiple straps 1202 that can be manually wrapped together using hook and loop fasteners 1204. RIPCD 1300, see FIG. 13, includes a controller 1302 and multiple straps 1304 interconnected with webbing portion 1306. Latch portions 1308 may also be used to interconnect the straps 1304. In another embodiment, the straps used with RIPCDs 700, 800, 900, 1000, 1100, 1200 and 1300 are not coiled but rather form generally circular shaped pressure patterns on the patient's arm.

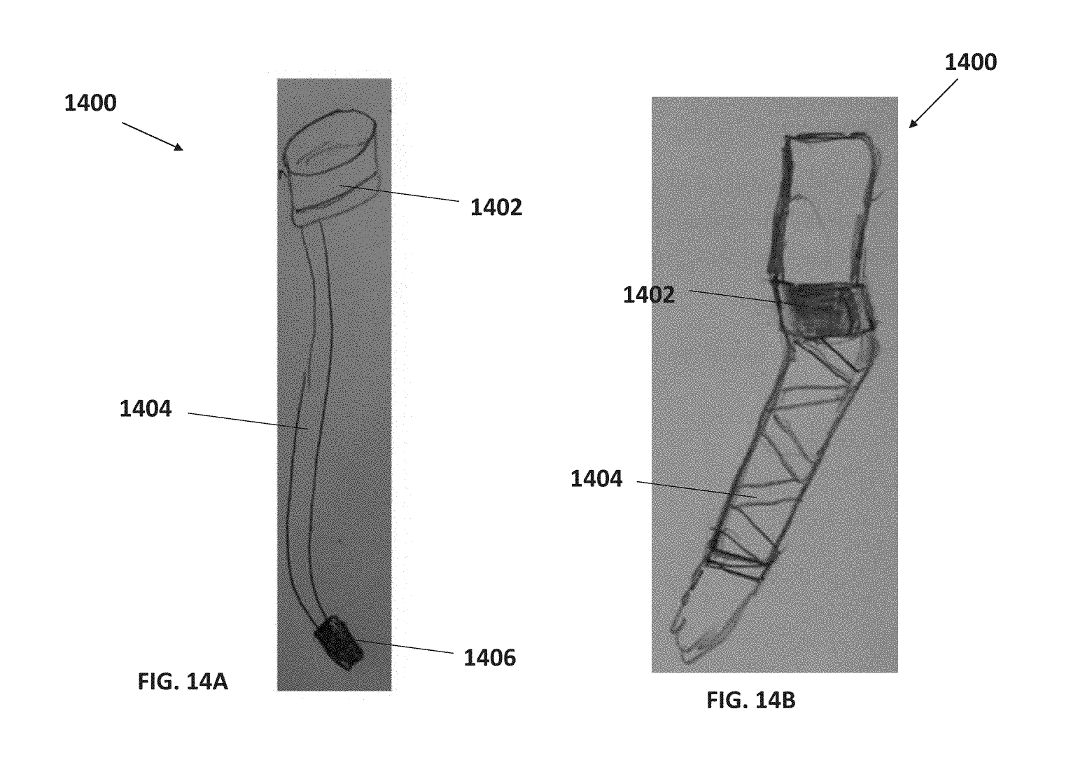

FIGS. 14A and 14B, illustrate RIPCD 1400 which includes a first compression portion, 1402 in the form of a bicep cuff, that is selectively operable to apply a coil shaped pressure pattern around at least 360 degrees of the upper arm portion (the bicep portion), a second compression portion 1402, in the form of a strap, that is selectively wrappable about the arm to apply a coil shaped pressure pattern around at least 360 degrees of the lower arm portion (the forearm portion) and a controller 1406 that operates the compression portions using remote ischemic preconditioning in order to: 1) reduce the blood flow in the upper and lower arm portions for at least 30 continuous minutes; and 2) increase blood flow in the patient as a result of remote ischemic preconditioning. In one embodiment, both compression portions 1402, 1404 selectively apply (and remove) pressure using pneumatic inflation.

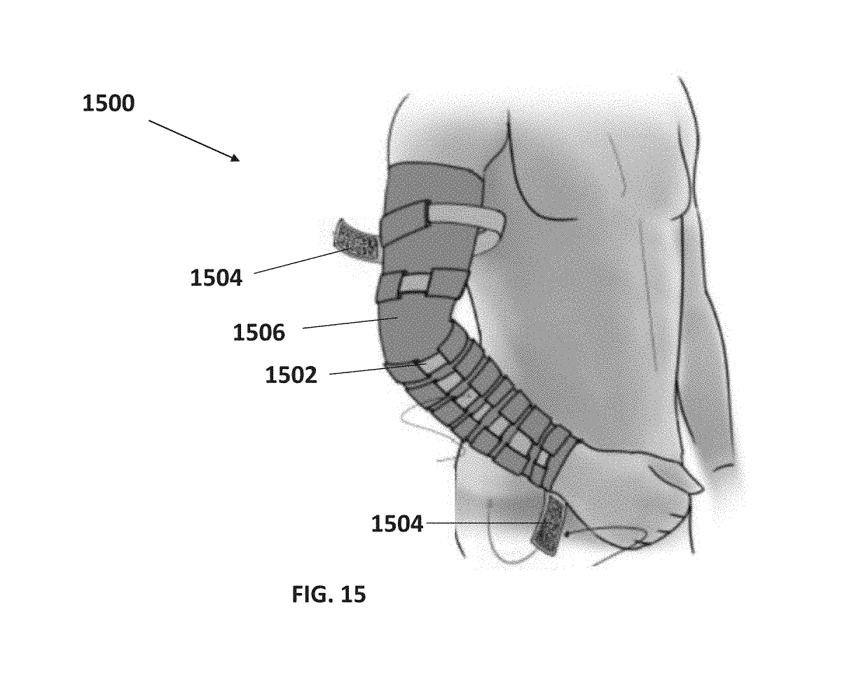

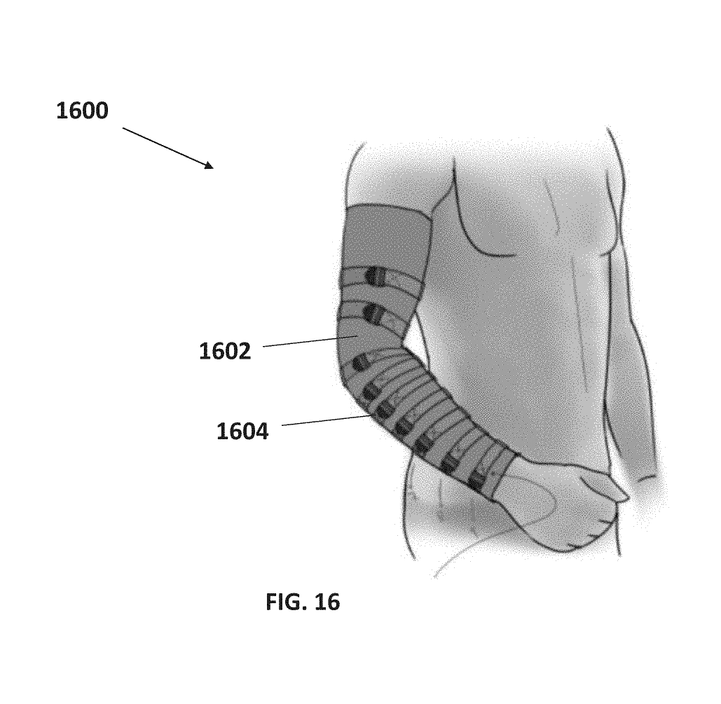

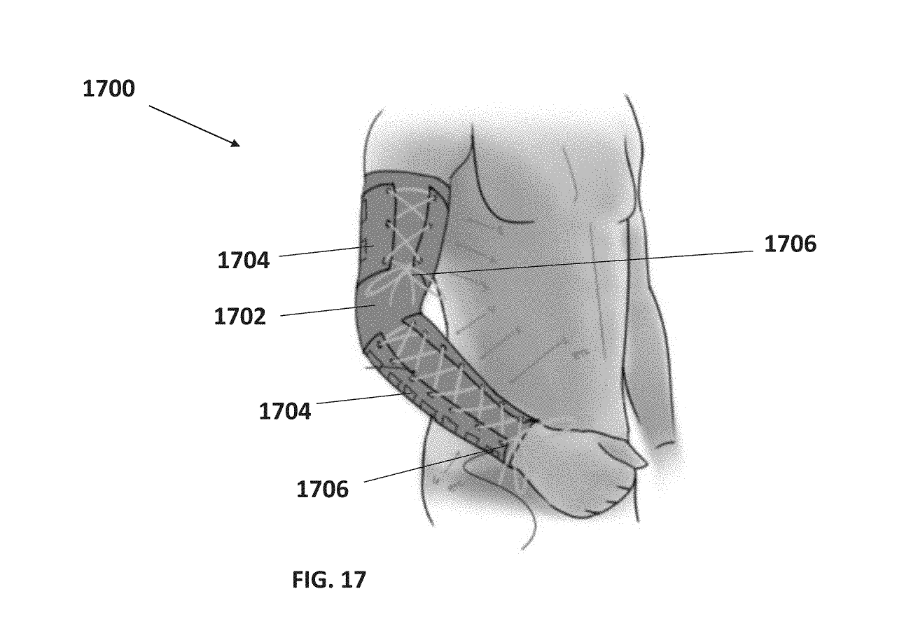

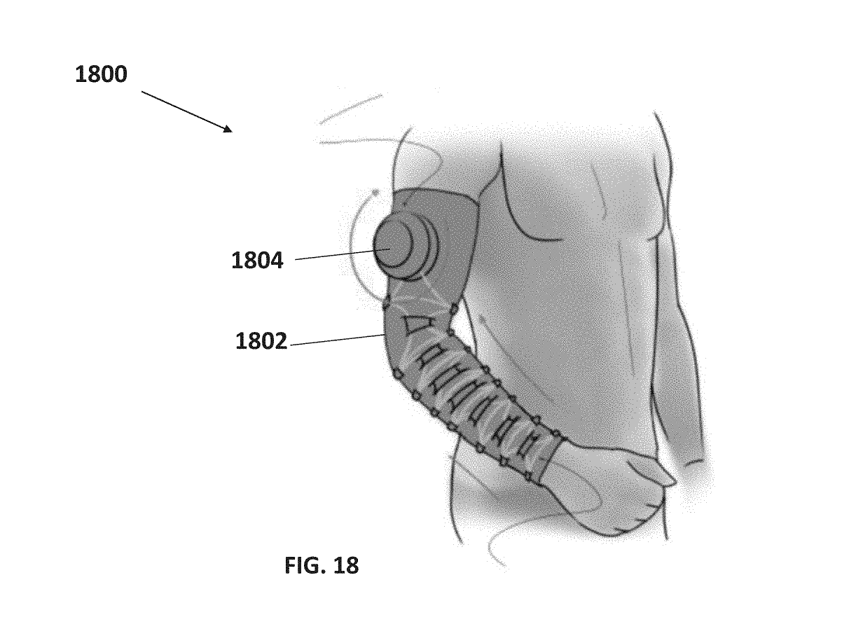

FIGS. 15-18, illustrate RIPCDs 1500, 1600, 1700 and 1800 that include a first compression portion that is selectively operable to apply a circular or coil shaped pressure pattern around at least 360 degrees of the upper arm portion (the bicep portion) and a second compression portion that is selectively operable to apply a circular or coil shaped pressure pattern around at least 360 degrees of the lower arm portion (the forearm portion). These devices, however, use only manual devices to apply the required pressure to use remote ischemic preconditioning in order to: 1) reduce the blood flow in the upper and lower arm portions for at least 30 continuous minutes; and 2) increase blood flow in the patient as a result of remote ischemic preconditioning. RIPCD 1500, see FIG. 15, uses straps 1502 that may use elastic or non-elastic materials that can be applied using hook and loop fastening techniques and/or buckles 1504. The straps 1502 may be interconnected with a sleeve 1506, as shown. RIPCD 1600, see FIG. 16, includes a sleeve 1602 and uses pull band straps 1602 to create the necessary pressure. RIPCD 1700, see FIG. 17, includes a sleeve 1702 and uses one upper and one lower strap 1704 that are tightened using laces 1706, similar to shoelaces. RIPCD 1800, see FIG. 18, includes a sleeve 1802 and uses a BOA® system 1804 to create the necessary pressure.

In many embodiments described above, one or more straps are used. The straps may have arm contact surfaces that apply the pressure pattern to the patient's arm. In some embodiments, the arm contact surface of the strap has a width of 1.0 inch or less. In another embodiment, the arm contact surface of the strap has a width of 0.75 inches or less. In another embodiment, the arm contact surface of the strap has a width of 0.5 inches or less.

In the patent claims that follow, it should be understood that any component referred to as being “associated” is not being claimed positively but rather indicates the environment in which the claimed invention is used. Thus, for one non-limiting example, if a patent claim includes “an associated patient” then Applicant's intent is that infringement does not require an actual patient or patient's limb. Rather, infringement only requires the device or method can be used with a patient.

Having thus described the invention, it is now claimed:

I/We claim:

1. A remote ischemic preconditioning application device for use with an associated patient having an arm with an upper arm portion and a lower arm portion; the device comprising:
a first compression portion that is selectively operable to apply a coil shaped pressure pattern around at least 360 degrees of the upper arm portion;
a second compression portion that is selectively operable to apply a coil shaped pressure pattern around at least 360 degrees of the lower arm portion; and
a controller that operates the first and second compression portions using remote ischemic preconditioning to:
1) reduce blood flow in the upper and lower arm portions for at least 30 continuous minutes; and
2) increase blood flow in the patient as a result of remote ischemic preconditioning.

2. The remote ischemic preconditioning application device of claim 1 wherein:
the first compression portion is selectively operable to apply the coil shaped pressure pattern as a generally helically shaped pressure pattern around the upper arm portion for at least 360 degrees; and
the second compression portion is selectively operable to apply the coil shaped pressure pattern as a generally helically shaped pressure pattern around the lower arm portion for at least 1440 degrees.

3. The remote ischemic preconditioning application device of claim 1 wherein:
the first compression portion is selectively inflatable to apply the coil shaped pressure pattern around the upper arm portion; and
the second compression portion is selectively inflatable to apply the coil shaped pressure pattern around the lower arm portion.

4. The remote ischemic preconditioning application device of claim 1 wherein:
the first compression portion is selectively operable to apply the coil shaped pressure pattern around the upper arm portion using only sub-occlusive pressure; and
the second compression portion is selectively operable to apply the coil shaped pressure pattern around the lower arm portion using only sub-occlusive pressure.

5. The remote ischemic preconditioning application device of claim 1 wherein:
the first compression portion is a bicep cuff; and
the controller is operable using the bicep cuff and a pressure sensor to determine systolic and diastolic blood pressure of the associated patient's arm.

6. The remote ischemic preconditioning application device of claim 1 wherein:
the controller operates the first and second compression portions using remote ischemic preconditioning to reduce blood flow in the upper and lower arm portions simultaneously.

7. The remote ischemic preconditioning application device of claim 1 wherein:
the controller operates the first and second compression portions using remote ischemic preconditioning to reduce blood flow in the upper and lower arm portions sequentially.

8. A method for use in applying remote ischemic preconditioning to an associated patient having an arm with an upper arm portion and a lower arm portion; the method comprising the steps of:
A) providing a remote ischemic preconditioning application device comprising:
1) a first compression portion that is selectively operable to apply a coil shaped pressure pattern around at least 360 degrees of the upper arm portion;
2) a second compression portion that is selectively operable to apply a coil shaped pressure pattern around at least 360 degrees of the lower arm portion; and
3) a controller that operates the first and second compression portions;
B) providing the remote ischemic preconditioning application device to be operable to perform the steps of:
1) reducing blood flow in the upper and lower arm portions for at least 30 continuous minutes; and
2) increasing blood flow in the patient as a result of remote ischemic preconditioning.

9. The method of claim 8 wherein step B1 comprises the step of:
reducing blood flow in the upper and lower arm portions simultaneously.

10. The method of claim 8 wherein step B1 comprises the step of:
reducing blood flow in the upper and lower arm portions sequentially.

11. The method of claim 8 wherein step B1 comprises the step of:
reducing blood flow in the upper and lower arm portions using only sub-occlusive pressure.

12. The method of claim 8 wherein:
step A1 comprises the step of: providing the first compression portion to be selectively inflatable to apply the coil shaped pressure pattern as a generally helically shaped pressure pattern around the upper arm portion; and
step A2 comprises the step of: providing the second compression portion to be selectively inflatable to apply the coil shaped pressure pattern as a generally helically shaped pressure pattern around the lower arm portion.

13. The method of claim 12 further comprising the steps of:
providing the first compression portion to be a bicep cuff;
providing a pressure sensor; and
providing the controller to be operable using the bicep cuff and the pressure sensor to determine systolic and diastolic blood pressure of the associated patient's arm.

14. The method of claim 13 wherein the controller is operable to:
calculate the mean arterial pressure from the blood pressure;
re-inflate the bicep cuff to achieve and maintain the mean arterial pressure; and
deflate the bicep cuff after 30 continuous minutes.

15. A remote ischemic preconditioning application device for use with an associated patient having an arm with an upper arm portion and a lower arm portion; the device comprising:
a bicep cuff that is selectively operable to apply a coil shaped pressure pattern around at least 360 degrees of the upper arm portion;
a forearm strap that extends from the bicep cuff and that is selectively operable to apply a coil shaped pressure pattern around at least 360 degrees of the lower arm portion; and
a controller that operates the bicep cuff and the forearm strap using remote ischemic preconditioning to:
1) reduce blood flow in the upper and lower arm portions using only sub-occlusive pressure; and
2) increase blood flow in the patient as a result of remote ischemic preconditioning.

16. The remote ischemic preconditioning application device of claim 15 wherein:
the bicep cuff is selectively operable to apply the coil shaped pressure pattern as a generally helically shaped pressure pattern around at least 720 degrees of the upper arm portion; and
the forearm strap is selectively operable to apply the coil shaped pressure pattern as a generally helically shaped pressure pattern around at least 1440 degrees of the lower arm portion.

17. The remote ischemic preconditioning application device of claim 16 wherein:
the bicep cuff is selectively inflatable to apply the generally helically shaped pressure pattern around the upper arm portion; and
the forearm strap is selectively inflatable to apply the generally helically shaped pressure pattern around the lower arm portion.

18. The remote ischemic preconditioning application device of claim 17 wherein:
the bicep cuff comprises an arm contact surface that is selectively operable to apply the generally helically shaped pressure pattern around the upper arm portion;
the forearm strap comprises an arm contact surface that is selectively operable to apply the generally helically shaped pressure pattern around the lower arm portion;
the arm contact surface of the bicep cuff has a width of 1.0 inches or less; and
the arm contact surface of the forearm strap has a width of 1.0 inches or less.

19. The remote ischemic preconditioning application device of claim 18 wherein:
the controller is operable using the bicep cuff and a pressure sensor to determine systolic and diastolic blood pressure of the associated patient's arm.

20. The remote ischemic preconditioning application device of claim 19 wherein:
the controller operates the bicep cuff and the forearm strap using remote ischemic preconditioning to reduce blood flow in the upper and lower arm portions for at least 30 continuous minutes.
